(19) United States
(12) Patent Application Publication
(10) Pub. No.: US 2025/0186756 A1
(43) Pub. Date: Jun. 12, 2025

(54) PORT ACCESS-LOCK DEVICE AND SYSTEM

(71) Applicant: H. Lee Moffitt Cancer Center and Research Institute, Inc., Tampa, FL (US)

(72) Inventor: Michael A. Vogelbaum, Tampa, FL (US)

(73) Assignee: H. Lee Moffitt Cancer Center and Research Institute, Inc., Tampa, FL (US)

(21) Appl. No.: 18/846,637

(22) PCT Filed: Mar. 9, 2023
(86) PCT No.: PCT/US2023/014884

§ 371 Date: Sep. 12, 2024

Related U.S. Application Data
(60) Provisional application No. 63/320,492, filed on Mar. 16, 2022.

(51) Int. Cl. A61M 39/02

(52) U.S. Cl. CPC A61M 39/0247; A61M 2039/025; A61M 2039/0267; A61M 2039/0276; A61M 2039/0294; A61M 2205/0288

(57) ABSTRACT

Various implementations include a system for coupling a needle to a port. The system includes a port and a needle. The port includes a reservoir and a lock box disposed within the cavity and defining a lock opening. The reservoir includes a membrane and a base coupled to the membrane, which together define a cavity. The reservoir is configured to be implanted under the skin of a person. The needle defines a lumen extending along a needle longitudinal axis. The needle includes a key disposed within the lumen. The key is movable from a first position to a second position. A portion of the key extends further in a radially outward direction in the second position than in the first position. A distal end of the needle is disposable within the lock opening such that the key is couplable to the lock box when in the second position.

### BACKGROUND

Currently, there are multiple therapies for brain tumors that provide reliable responses, none of which are curative. One of the limiting factors for systemically administered therapeutics is the presence of the blood-brain-barrier (BBB), which excludes most therapeutics from reaching the central nervous system.

An alternative approach is to delivery therapies directly into brain tumors or tumor infiltrated brain tissue. Direct brain delivery requires the use of a delivery system. There are no delivery devices currently that are specifically indicated for delivery of a therapeutic to brain tissue (only to the cerebrospinal fluid spaces). This is because there are no therapeutics yet approved for direct brain delivery.

Most oncology therapeutics require multiple cycles of administration, often lasting more than six months in duration and sometimes even over multiple years. Placement of a delivery device in the brain requires a neurosurgical procedure, typically under general anesthesia. This requirement makes it virtually impossible to develop therapeutics that require a prolonged period of intermittent administration to the brain, as two of the currently available devices can be used in the operating room only, and the other two cannot be left in place for more than a few days.

One challenge that will arise with use of a brain access device is that the infusions are likely to occur over hours to possibly days. Typically, and for on-label treatment to the ventricles, current port devices are accessed with a needle for a period of minutes and the needle is held in place manually. The risk of needle dislodgement during infusions over hours to days is very high, and so a conventional reservoir is not an acceptable design for the type of use envisioned herein.

This problem is also encountered with intravenous port systems, typically placed on the chest wall. For that application, there are multiple external fixation devices which hold the access needle in place. These devices typically adhere to the skin that surrounds the port access point. External fixation is a challenging approach for a port located in the scalp as adherence to scalp is more challenging due to secretions and hair.

Thus, there is a need for a fully implanted system designed to be accessed intermittently for prolonged (hours to days) periods of time.

### BRIEF DESCRIPTION OF THE DRAWINGS

Example features and implementations are disclosed in the accompanying drawings. However, the present disclosure is not limited to the precise arrangements and instrumentalities shown.

FIGS. 1A-1C show a side view of a system for coupling a needle to a port, according to one implementation.

FIGS. 2A-2C show a top view, an end view, and a side view of the needle coupling to the lock box of the system of FIGS. 1A-1C.

FIGS. 3A-3C show side views of the key being extended from the needle of FIGS. 1A-1C.

FIG. 4A shows a detailed view of the needle of FIGS. 1A-1C.

FIG. 4B shows a cross-sectional view of the needle of FIG. 4A along lines B-B.

FIG. 4C shows a cross-sectional view of the needle of FIG. 4B along lines C-C.

FIG. 5 shows side views of the key being extended from the needle, according to another implementation.

FIG. 6 show a top view, an end view, and a side view of a lock box, according to another implementation.

FIGS. 7A-7C show a side view of a system for coupling a needle to a port, according to another implementation.

FIGS. 8A-8C show a top view and an end view of the needle coupling to the lock box of the system of FIGS. 7A-7C.

FIGS. 9A and 9B show side views of the expandable volume being extended from the needle of FIGS. 7A-7C.

FIGS. 10A-10C show a side view of a system for coupling a needle to a port, according to another implementation.

FIGS. 11A-11C show a side view of a system for coupling a needle to a port, according to another implementation.

FIGS. 12A and 12B show top views of a system for localizing a port, according to one implementation.

### SUMMARY

Various implementations include a system for coupling a needle to a port. The system includes a port and a needle. The port includes a reservoir including a membrane and a base coupled to the membrane, the membrane and the base defining a cavity. The reservoir is configured to be implanted under the skin of a person with the membrane facing outwardly from the person. The port further includes a lock box disposed within the cavity, the lock box defining a lock opening. The needle includes a needle longitudinal axis and a distal end, the needle defining a lumen extending along the needle longitudinal axis. The needle includes a key disposed within the lumen, the key being movable from a first position to a second position. A portion of the key extends further in a radially outward direction relative to the needle longitudinal axis in the second position than in the first position. The distal end of the needle is disposable within the lock opening such that the key is couplable to the lock box when in the second position.

In some implementations, the lock box is coupled to the base.

In some implementations, the membrane is penetrable by the distal end of the needle.

In some implementations, the lock opening includes a first opening portion and a second opening portion. The first opening portion is directly open to a surface of the lock box and the second opening portion is directly open to the first opening portion. The first opening portion is shaped to accept the distal end of the needle when the key is in the second position. The second opening portion is shaped such that, when the distal end of the needle is disposed within the second opening portion, rotation of the needle in the second position about the needle longitudinal axis couples the needle to the lock box.

In some implementations, the lock opening includes a first opening portion and a second opening portion. The first opening portion is directly open to a surface of the lock box and the second opening portion is directly open to the first opening portion. The first opening portion is shaped to accept the distal end of the needle when the key is in the first position. The second opening portion is shaped such that, when the distal end of the needle is disposed within the second opening portion, movement of the key from the first position to the second position couples the needle to the lock box.

In some implementations, the key has a key longitudinal axis and includes a first key portion, a second key portion spaced apart from the first key portion along the key longitudinal axis, and a hinging portion disposed along the key longitudinal axis between the first key portion and the second key portion. The second key portion rotates radially outwardly about the hinging portion when the key is moved from the first position to the second position.

In some implementations, the hinging portion includes a resilient member.

In some implementations, the resilient member is biased toward the second position and is urgable toward the first position.

In some implementations, the resilient member includes a spring.

In some implementations, the key is axially slidable within the lumen of the needle from a retracted position to an extended position. At least a portion of the second key portion is disposed within the lumen in the retracted position, and the second key portion and at least a portion of the hinging portion is disposed external from the lumen in the extended position.

In some implementations, the needle further includes a slider fixedly coupled to the key for sliding the key from the retracted position to the extended position.

In some implementations, the key includes an expandable volume. The expandable volume extends further in a radially outward direction relative to the needle longitudinal axis in the second position than in the first position.

In some implementations, the expandable volume includes a bladder in fluid communication with a fluid source. The expandable volume is movable from the first position to the second position by causing fluid from the fluid source to flow into the bladder.

In some implementations, the needle further includes a slider coupled the fluid source for causing from the fluid source to flow into the bladder.

In some implementations, the needle includes two or more keys.

In some implementations, the needle defines one or more radial efflux ports extending from an outer surface of the needle to the lumen.

In some implementations, the one or more radial efflux ports of the needle are disposed within the cavity and external to the lock opening when the needle is coupled to the lock box.

Various other implementations include a system for coupling a needle to a port. The system includes a port and a needle. The port includes a reservoir including a membrane and a base coupled to the membrane, the membrane and the base defining a cavity. The reservoir is configured to be implanted under the skin of a person with the membrane facing outwardly from the person. The needle includes a needle longitudinal axis and a distal end, the needle defining a lumen extending along the needle longitudinal axis. The needle includes a key disposed within the lumen, the key being movable from a first position to a second position. A portion of the key extends further in a radially outward direction relative to the needle longitudinal axis in the second position than in the first position.

In some implementations, the key has a key longitudinal axis and includes a first key portion, a second key portion spaced apart from the first key portion along the key longitudinal axis, and a hinging portion disposed along the key longitudinal axis between the first key portion and the second key portion. The second key portion rotates radially outwardly about the hinging portion when the key is moved from the first position to the second position.

In some implementations, the hinging portion includes a resilient member, the resilient member being biased toward the second position and is urgable toward the first position.

In some implementations, the key is axially slidable within the lumen of the needle from a retracted position to an extended position. At least a portion of the second key portion is disposed within the lumen in the retracted position. The second key portion and at least a portion of the hinging portion is disposed external from the lumen in the extended position.

In some implementations, the needle further includes a slider fixedly coupled to the key for sliding the key from the retracted position to the extended position.

In some implementations, the key includes an expandable volume. The expandable volume extends further in a radially outward direction relative to the needle longitudinal axis in the second position than in the first position.

In some implementations, the expandable volume includes a bladder in fluid communication with a fluid source. The expandable volume is movable from the first position to the second position by causing fluid from the fluid source to flow into the bladder. The needle further includes a slider coupled the fluid source for causing from the fluid source to flow into the bladder.

In some implementations, the needle includes two or more keys.

In some implementations, wherein the needle defines one or more radial efflux ports extending from an outer surface of the needle to the lumen.

Various other implementations include a system for locating an implanted port. The system includes a port and a port localizer. The port includes a reservoir including a membrane and a base coupled to the membrane, the membrane and the base defining a cavity. The reservoir is configured to be implanted under the skin of a person with the membrane facing outwardly from the person. The port further includes a paramagnetic localizing disc disposed in a central portion of the base. The port localizer device is configured to detect the location of the paramagnetic localizing disc. The port localizer device includes a transmitter/receiver coil defining an opening and a detachable handle couplable to and in electrical communication with the transmitter/receiver coil.

In some implementations, the port further includes a lock box disposed within the cavity, the lock box defining a lock opening.

In some implementations, the system further includes a needle having a needle longitudinal axis and a distal end, the needle defining a lumen extending along the needle longitudinal axis. The needle includes a key disposed within the lumen, the key being movable from a first position to a second position. A portion of the key extends further in a radially outward direction relative to the needle longitudinal axis in the second position than in the first position. The distal end of the needle is disposable within the lock opening such that the key is couplable to the lock box when in the second position.

In some implementations, the needle is disposable with the opening of the transmitter/receiver coil.

### DETAILED DESCRIPTION

The devices, systems, and methods disclosed herein provide for a mechanism that allows a port access needle to remain in place for extended periods of time, and to be detected when the port is not visible. The mechanism allows a needle to be locked in place thereby preventing inadvertent needle dislodgement. While there are needle securing devices in use today, these are external to the needle itself and generally are placed over the needle and adhere to the surrounding skin. Certain locations (e.g. scalp) are less amenable to adhesion-based approaches and an internal locking mechanism may be more optimal.

By “extended period of time” is meant that the port can be in place for hours, such as 12, 24, 36, or 48 hours or more, or days, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 days, or 1, 2, 3, 4, 5, 6, 7, 8, 9, 10, 11, or 12 months, or 1, 2, 3, 4, 5, 6, 7, 8,9, or 10 years or more, or any amount above, below, or in-between these amounts.

By “not visible” is meant that the port, which is located beneath the skin, does not cause significant rise in the skin and cannot be easily seen visibly.

The devices and methods disclosed herein can be used in a variety of manners, but specifically contemplated is their use with an implantable port. Such implantable ports are known to those of skill in the art, and are exemplified by U.S. Pat. Nos. 10,485,964, 9,474,888, 8,147,455, 8,932,271, and 9,895,523, all of which are incorporated by reference herein in their entirety for their teaching concerning ports. Specifically contemplated herein are devices and methods for use with ports which are implanted in the skull.

Various implementations include a system for coupling a needle to a port. The system includes a port and a needle. The port includes a reservoir and a lock box disposed within the cavity and defining a lock opening. The reservoir includes a membrane and a base coupled to the membrane, which together define a cavity. The reservoir is configured to be implanted under the skin of a person. The needle defines a lumen extending along a needle longitudinal axis. The needle includes a key disposed within the lumen. The key is movable from a first position to a second position. A portion of the key extends further in a radially outward direction in the second position than in the first position. A distal end of the needle is disposable within the lock opening such that the key is couplable to the lock box when in the second position.

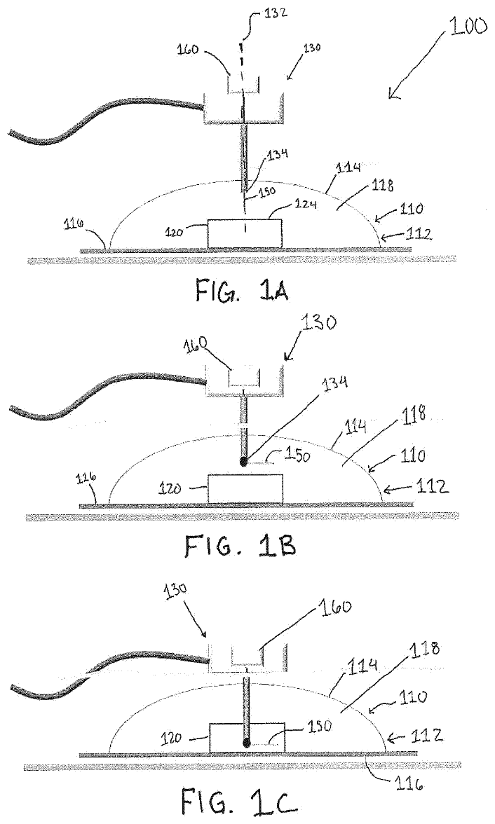

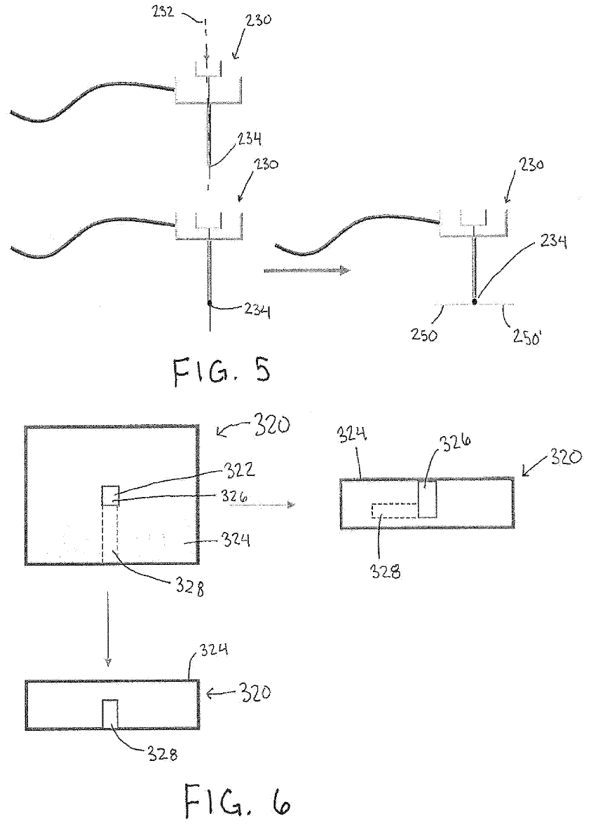

FIGS. 1A-4C show a system 100 for coupling a needle 130 to a port 110, according to aspects of some implementations. The system 100 includes a port 110 and a needle 130.

The port 110 includes a reservoir 112 and a lock box 120. The reservoir 112 includes a membrane 114 and a base 116 coupled to the membrane 114. The membrane 114 and the base 116 define a cavity 118. The membrane 114 is made of a material that is capable of being penetrated by a needle but is resilient enough to close the opening created by the needle 130 once the needle 130 is removed. The reservoir 112 is configured to be implanted under the skin of a person with the membrane 114 facing outwardly from the person.

The lock box 120 is disposed within the cavity 118 and defines a lock opening 122. The lock box 120 is coupled to the base 116.

The needle 130 has a needle longitudinal axis 132 and a distal end 134. The needle 130 defines a lumen 136 extending along the needle longitudinal axis 132. The needle 130 defines one or more radial efflux ports 140 extending from an outer surface of the needle 138 to the lumen 136.

The needle 130 further includes a key 150 disposed within the lumen 136. The key 150 is movable from a first position (shown in FIG. 1A) to a second position (shown in FIGS. 1B and 1C). A portion of the key extends further in a radially outward direction relative to the needle longitudinal axis 132 in the second position than in the first position. The distal end 134 of the needle 130 is disposable within the lock opening 122 such that the key 150 is couplable to the lock box 120 when in the second position.

The key 150 shown in FIGS. 1A-4C has a key longitudinal axis 152 and includes a first key portion 154, a second key portion 156 spaced apart from the first key portion 154 along the key longitudinal axis 152, and a hinging portion 158 disposed along the key longitudinal axis 152 between the first key portion 154 and the second key portion 156. The second key portion 156 rotates radially outwardly about the hinging portion 158 when the key 150 is moved from the first position to the second position.

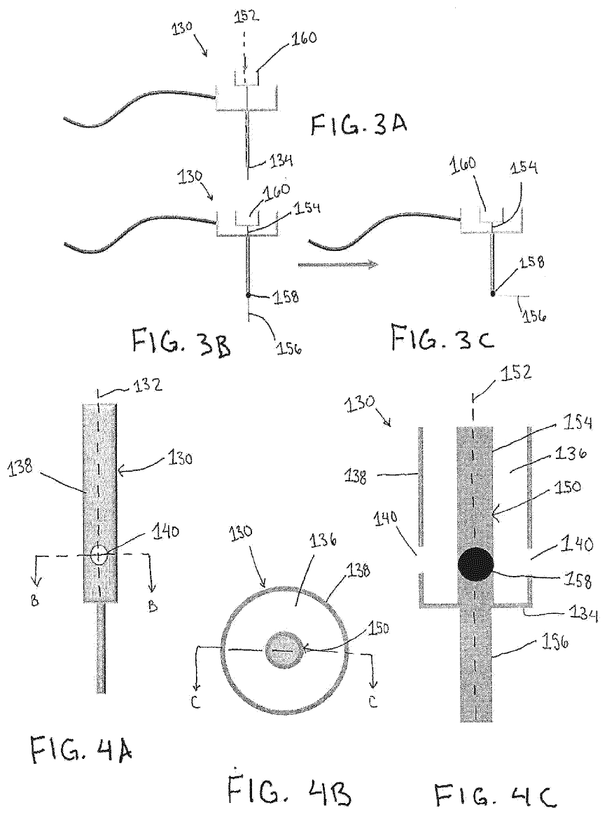

The hinging portion 158 of the key 150 shown in FIG. 1A-4C includes a resilient member (e.g., a spring) that is biased toward the second position and is urgable toward the first position. The key 150 is axially slidable within the lumen 136 of the needle 130 from a retracted position (shown in FIG. 3A) to an extended position (FIG. 3B). At least a portion of the second key portion 156 is disposed within the lumen 136 in the retracted position. The second key portion 156 and at least a portion of the hinging portion 158 is disposed external from the lumen 136 in the extended position.

The needle 130 shown in FIG. 1A-4C further includes a slider 160 fixedly coupled to the key 150 for sliding the key 150 from the retracted position to the extended position. The slider 160 shown in FIGS. 1A-4C is a button, but in other implementations, the slider can be any mechanism capable of moving the key from the retracted position to the extended position.

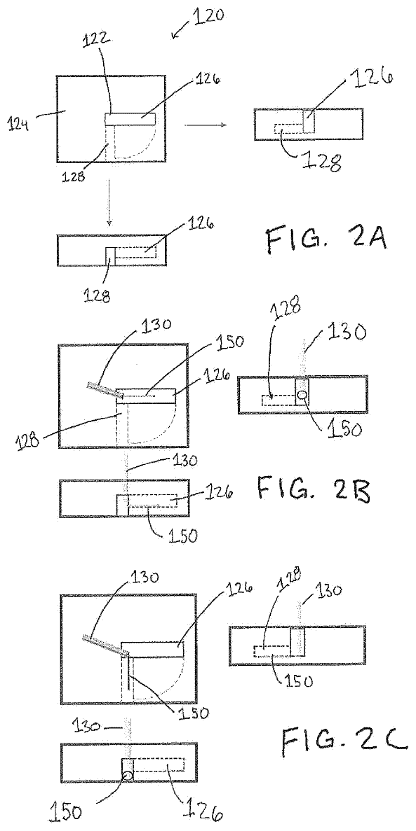

The lock opening 122 of the lock box 120 shown in FIG. 1A-2C includes a first opening portion 126 and a second opening portion 128. The first opening portion 126 is directly open to a top surface of the lock box 124, and the second opening portion 128 is directly open to the first opening portion 126.

The first opening portion 126 is shaped to accept the distal end 134 of the needle 130 when the key 150 is in the second position. The second opening portion 128 is shaped such that, when the distal end 134 of the needle 130 is further inserted into the lock opening 122 such that it is disposed within the second opening portion 128, rotation of the needle 130 in the second position about the needle longitudinal axis 132 couples the needle 130 to the lock box 120. In this coupled position, the one or more radial efflux ports 140 of the needle 130 are disposed within the cavity 118 of the reservoir 112 and external to the lock opening 122.

Rotation of the needle 130 about the needle longitudinal axis 132 in the opposite direction decouples the needle 130 from the lock box 120. The key 150 can then be moved from the second position to the first position such that the needle 130 can be removed through the membrane 114 and out of the reservoir 112 of the port 110.

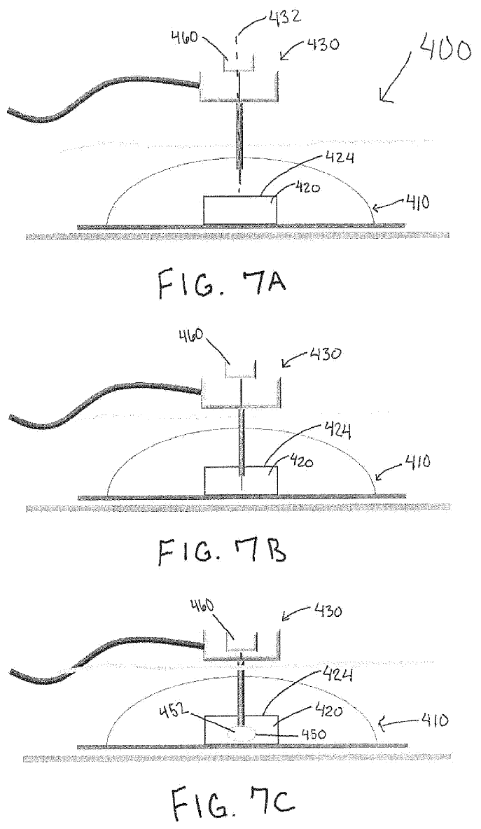

FIG. 5 shows a needle 230 according to another implementation. The needle 230 shown in FIG. 5 is similar to the needle 130 shown in FIGS. 1A-4C, but the needle 230 shown in FIG. 5 includes two keys 250, 250′. When the keys 250, 250′ are moved from the first position to the second position, both keys 250, 250′ extend radially outwardly in different radial directions. A lock box to be used with this needle would include a first opening portion that is shaped to accept the distal end 234 of the needle 230 when both keys 250, 250′ are in the second position. The second opening portion would be shaped such that, when the distal end 234 of the needle 230 is further inserted into the lock opening such that it is disposed within the second opening portion, rotation of the needle 230 in the second position about the needle longitudinal axis 232 couples the needle 230 to the lock box. It will be appreciated that, in some implementations, a needle can include any number of keys, and the lock box can include a first opening portion and a second opening portion that accommodate the keys of the needle.

FIG. 6 shows another implementation of a lock box 320 that can be used with the same needle 130 as shown in FIGS. 1A-4C. In this implementation, the lock opening 322 includes a first opening portion 326 and a second opening portion 328. The first opening portion 326 is directly open to a top surface of the lock box 324, and the second opening portion 328 is directly open to the first opening portion 326. The first opening portion 326 is shaped to accept the distal end of the needle when the key is still in the first position. The second opening portion 328 is shaped such that, when the distal end of the needle is further inserted into the lock opening 322 such that it is disposed within the second opening portion 328, movement of the key from the first position to the second position couples the needle to the lock box 320. Movement of the key from the second position to the first position decouples the needle from the lock box 320.

FIGS. 7A-9B show a system 400 for coupling a needle 430 to a port 410 according to another implementation. The needle 430 shown in FIG. 7A-9B includes an expandable volume 450. The expandable volume 450 includes a bladder 452 in fluid communication with a fluid source. The expandable volume 450 extends further in a radially outward direction relative to the needle longitudinal axis 432 in the second position than in the first position. The expandable volume 450 is movable from the first position to the second position by causing fluid from the fluid source to flow into the bladder 452. Like the needle 130 shown in FIGS. 1A-4C, the needle 430 includes a slider 460. However, the slider 460 shown in FIG. 7A-9B is coupled to the fluid source for causing fluid from the fluid source to flow into the bladder 452.

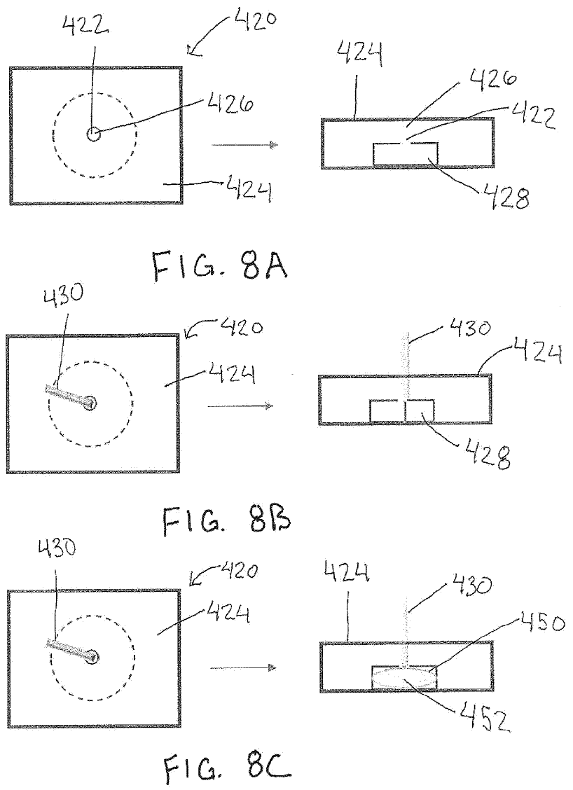

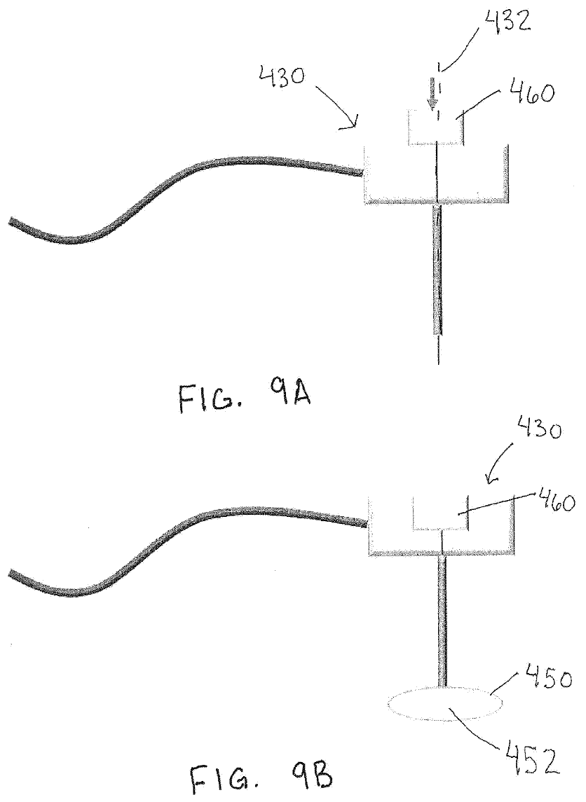

FIGS. 8A-8C show another implementation of a lock box 420 that can be used with the needle 430 in FIGS. 7A-9B. In this implementation, the lock opening 422 includes a first opening portion 426 and a second opening portion 428. The first opening portion 426 is directly open to a top surface of the lock box 424, and the second opening portion 428 is directly open to the first opening portion 426. The first opening portion 426 is shaped to accept the distal end of the needle 430 when the expandable volume 450 is still in the first position. The second opening portion 428 is shaped such that, when the distal end of the needle 430 is further inserted into the lock opening 422 such that it is disposed within the second opening portion 428, movement of the expandable volume 450 from the first position to the second position couples the needle 430 to the lock box 420. Movement of the expandable volume 450 from the second position to the first position decouples the needle 430 from the lock box 420.

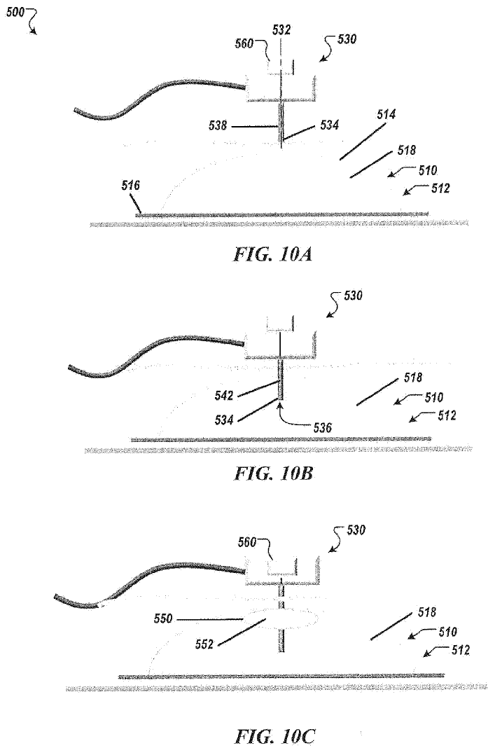

FIGS. 10A-10C show a system 500 for coupling a needle 530 to a port 510 according to another implementation. The port 510 includes a reservoir 512. The reservoir 512 includes a membrane 514 and a base 516 coupled to the membrane 514. The membrane 514 and the base 516 define a cavity 518. The membrane 514 is made of a material that is capable of being penetrated by a needle but is resilient enough to close the opening created by the needle 530 once the needle 530 is removed. The reservoir 512 is configured to be implanted under the skin of a person with the membrane 514 facing outwardly from the person.

The needle 530 shown in FIGS. 10A-10C has a needle longitudinal axis 532, a distal end 534, and a middle portion 542. The needle 530 defines a lumen 536 extending along the needle longitudinal axis 532. The needle 530 defines one or more radial efflux ports extending from an outer surface of the needle 538 to the lumen 536.

The needle 530 includes an expandable volume 550. The expandable volume 550 includes a bladder 552 in fluid communication with a fluid source. The expandable volume 550 extends further in a radially outward direction relative to the needle longitudinal axis 532 in the second position than in the first position. The expandable volume 550 is movable from the first position to the second position by causing fluid from the fluid source to flow into the bladder 552. Like the needle 130 shown in FIGS. 1A-4C, the needle 530 includes a slider 560. However, the slider 560 shown in FIG. 10A-10C is coupled to the fluid source for causing fluid from the fluid source to flow into the bladder 452.

In contrast to the system 400 of FIGS. 7A-9B, the system 500 of FIGS. 10A-10C does not include a lock box within the cavity 518. Rather, as shown in FIG. 10C, the expandable volume 550 is positioned at the middle portion 542 of the needle 530 (e.g., aligned with radial efflux ports along the outside surface 538 and at the middle portion 542 of the needle 530). Moving the expandable volume 550 from the first position to the second position couples the needle 530 to the cavity 518. Movement of the expandable volume 550 from the second position to the first position decouples the needle 530 from the cavity 518.

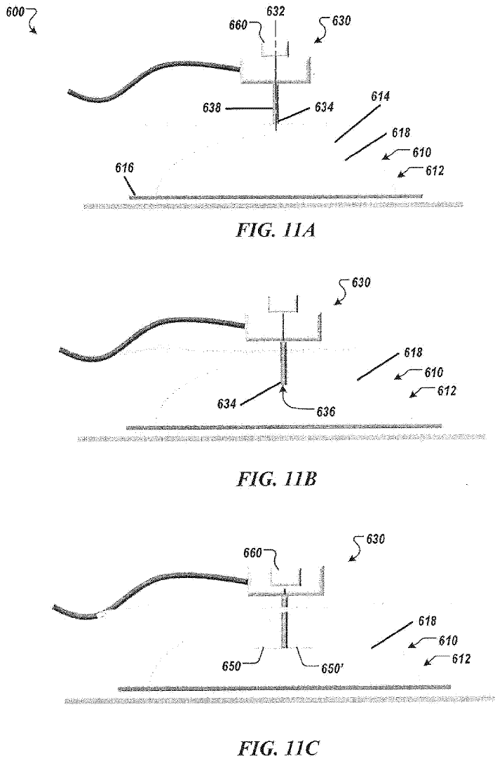

FIGS. 11A-11C show a system 600 for coupling a needle 630 to a port 610 according to another implementation. The port 610 includes a reservoir 612. The reservoir 612 includes a membrane 614 and a base 616 coupled to the membrane 514. The membrane 614 and the base 616 define a cavity 618.

The needle 630 has a needle longitudinal axis 632 and a distal end 634. The needle 630 defines a lumen 636 extending along the needle longitudinal axis 632. Similar to the needle 230 of FIG. 5, the needle 630 includes a snare comprising two prongs or keys 650, 650′. It will be appreciated that, in some implementations, a snare can include any number of prongs or keys (e.g., 3, 4, 5, 6, 8, 10, 15, or 20 keys disposed radially from needle longitudinal axis). The needle 630 shown in FIG. 11A-11C further includes a slider 660 fixedly coupled to the keys 650, 650′ for sliding the keys 650, 650′ from the retracted position and/or first position to the extended position and/or second.

Similar to system 500, system 600 does not include a lock box within the cavity 618. When the keys 650, 650′ are moved from the first position to the second position, both keys 650, 650′ extend radially outwardly in different radial directions such that the needle 650 cannot be easily removed from the port 610. Therefore, in the second position, the keys 650, 650′ couple to the needle 630 to the cavity 618.

In some implementations, each of the keys 650, 650′ include a first key portion, a second key portion spaced apart from the first key portion along the key longitudinal axis, and a hinging portion disposed along the key longitudinal axis between the first key portion and the second key portion, wherein the second key portion rotates radially outwardly about the hinging portion when the key is moved from the first position to the second position.

### Port Localizer

When a cranial reservoir is placed beneath the surface of the skin (e.g., on the surface of the skull), a bump is normally visible. The bump provides an easy way for medical professionals to identify the port and access it. However, a visible bump in a person's skin may be cosmetically unappealing, especially if multiple ports are implanted. To reduce cosmetic impact, a surgeon may recess the port further away from the skin surface. For example, the surgeon may drill a pocket in the skull into which the port fits. Thus, the top of the port sits at the surface of the skull and does not push into the scalp, removing the visible bump. One challenge with a recessed port is the accessibility and localization of the port. Without a visible bump, the port may be difficult to locate and/or access (e.g., for infusing medication).

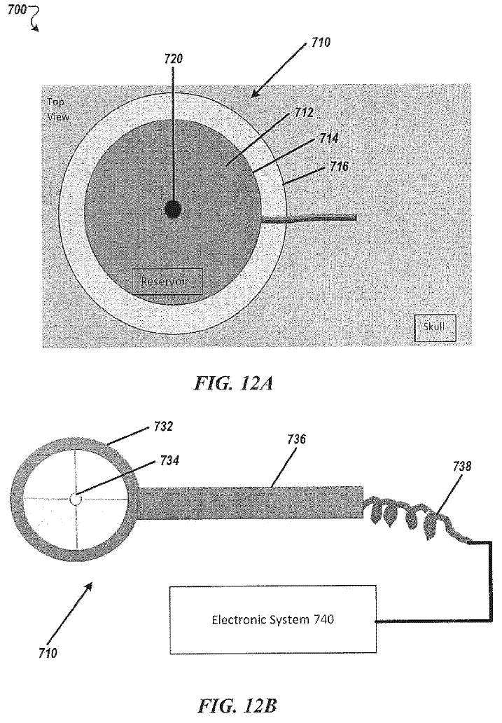

Provided herein are devices, systems, and methods for localizing a port (e.g., a recessed port implant). FIGS. 12A and 12B show a port localizer system 700 including a port 710 and a port localizer device 730. The port 710 (e.g., a port similar to those described above in FIGS. 1A-1C, 7A-7C, 10A-10C, and/or 11A-11C) includes a reservoir 712. The reservoir 712 includes a membrane 714 and a base 716 coupled to the membrane 714. The membrane 714 and the base 716 define a cavity 718. The membrane 714 is made of a material that is capable of being penetrated by a needle (e.g., a needle similar to those described above in FIGS. 1A-11C) but is resilient enough to close the opening created by the needle once the needle is removed.

The reservoir 712 is configured to be implanted under the skin of a person (e.g., implanted within a recess in the skull) with the membrane 714 facing outwardly from the person. The port 710 also includes a paramagnetic localizing disc 720 disposed in a central portion of the base 716.

The port localizer device 730 includes a transmitter/receiver coil 732 having an opening 734 disposed in a central portion of the transmitter/receiver coil 732. The port localizer device further includes a detachable handle 736 coupled to a wire 738. The handle 736 is configured to be removably attached to and in electrical communication with the transmitter/receiver coil 732.

In use, the transmitter/receiver coil 732 is coupled to the detachable handle 736, which, in turn, is coupled to an electronic system 740 (e.g., a power source and a controller/processor configured to send and/or receive signals from the port localizer device 730). The transmitter/receiver coil 732 is configured to detect (e.g., via electromagnetic signals) the presence and location of the paramagnetic localizing disc 720. The transmitter/receiver coil 732 is moved over a person's skin until the port localizer device 730 detects that the transmitter/receiver coil 732 is disposed directly over the paramagnetic localizing disc 720.

Once in place, a port access needle is placed through the opening 734 of the transmitter/receiver coil 732 such that the needle into the port 710. The needle can then lock in place and perform infusion procedures as needed. Once the needle is in place, the handle 736 can detach from the transmitter/receiver coil 732, leaving transmitter/receiver coil 732 in place above the port 710. Then, in some procedures, the handle 736 is attached to a different transmitter/receiver coil 732 to locating a second port 710. In some implementations, the transmitter/receiver coil 732 is disposable.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A system for coupling a needle to a port, the system comprising:
a port comprising:
a reservoir including a membrane and a base coupled to the membrane, the membrane and the base defining a cavity, wherein the reservoir is configured to be implanted under the skin of a person with the membrane facing outwardly from the person, and
a lock box disposed within the cavity, the lock box defining a lock opening; and
a needle having a needle longitudinal axis and a distal end, the needle defining a lumen extending along the needle longitudinal axis, wherein the needle includes a key disposed within the lumen, the key being movable from a first position to a second position, wherein a portion of the key extends further in a radially outward direction relative to the needle longitudinal axis in the second position than in the first position,
wherein the distal end of the needle is disposable within the lock opening such that the key is couplable to the lock box when in the second position.

2. The system of claim 1, wherein the lock box is coupled to the base.

3. The system of claim 1, wherein the membrane is penetrable by the distal end of the needle.

4. The system of claim 1, wherein the lock opening includes a first opening portion and a second opening portion, wherein the first opening portion is directly open to a surface of the lock box and the second opening portion is directly open to the first opening portion, wherein the first opening portion is shaped to accept the distal end of the needle when the key is in the second position, wherein the second opening portion is shaped such that, when the distal end of the needle is disposed within the second opening portion, rotation of the needle in the second position about the needle longitudinal axis couples the needle to the lock box.

5. The system of claim 1, wherein the lock opening includes a first opening portion and a second opening portion, wherein the first opening portion is directly open to a surface of the lock box and the second opening portion is directly open to the first opening portion, wherein the first opening portion is shaped to accept the distal end of the needle when the key is in the first position, wherein the second opening portion is shaped such that, when the distal end of the needle is disposed within the second opening portion, movement of the key from the first position to the second position couples the needle to the lock box.

6. The system of claim 1, wherein the key has a key longitudinal axis and includes a first key portion, a second key portion spaced apart from the first key portion along the key longitudinal axis, and a hinging portion disposed along the key longitudinal axis between the first key portion and the second key portion, wherein the second key portion rotates radially outwardly about the hinging portion when the key is moved from the first position to the second position.

7. The system of claim 6, wherein the hinging portion includes a resilient member.

8. The system of claim 7, wherein the resilient member is biased toward the second position and is urgable toward the first position.

9. The system of claim 8, wherein the resilient member includes a spring.

10. The system of claim 8, wherein the key is axially slidable within the lumen of the needle from a retracted position to an extended position, wherein at least a portion of the second key portion is disposed within the lumen in the retracted position, and wherein the second key portion and at least a portion of the hinging portion is disposed external from the lumen in the extended position.

11. The system of claim 10, wherein the needle further includes a slider fixedly coupled to the key for sliding the key from the retracted position to the extended position.

12. The system of claim 1, wherein the key includes an expandable volume, wherein the expandable volume extends further in a radially outward direction relative to the needle longitudinal axis in the second position than in the first position.

13. The system of claim 12, wherein the expandable volume includes a bladder in fluid communication with a fluid source, wherein the expandable volume is movable from the first position to the second position by causing fluid from the fluid source to flow into the bladder.

14. The system of claim 13, wherein the needle further includes a slider coupled the fluid source for causing from the fluid source to flow into the bladder.

15. The system of claim 1, wherein the needle includes two or more keys.

16. The system of claim 1, wherein the needle defines one or more radial efflux ports extending from an outer surface of the needle to the lumen.

17. The system of claim 16, wherein the one or more radial efflux ports of the needle are disposed within the cavity and external to the lock opening when the needle is coupled to the lock box.

18. A system for coupling a needle to a port, the system comprising:
a port comprising: a reservoir including a membrane and a base coupled to the membrane, the membrane and the base defining a cavity, wherein the reservoir is configured to be implanted under the skin of a person with the membrane facing outwardly from the person; and
a needle having a needle longitudinal axis and a distal end, the needle defining a lumen extending along the needle longitudinal axis, wherein the needle includes a key disposed within the lumen, the key being movable from a first position to a second position, wherein a portion of the key extends further in a radially outward direction relative to the needle longitudinal axis in the second position than in the first position.

19. The system of claim 18, wherein the key has a key longitudinal axis and includes a first key portion, a second key portion spaced apart from the first key portion along the key longitudinal axis, and a hinging portion disposed along the key longitudinal axis between the first key portion and the second key portion, wherein the second key portion rotates radially outwardly about the hinging portion when the key is moved from the first position to the second position.

20. The system of claim 19, wherein the hinging portion includes a resilient member, the resilient member being biased toward the second position and is urgable toward the first position.

21. The system of claim 20, wherein the key is axially slidable within the lumen of the needle from a retracted position to an extended position, wherein at least a portion of the second key portion is disposed within the lumen in the retracted position, and wherein the second key portion and at least a portion of the hinging portion is disposed external from the lumen in the extended position.

22. The system of claim 21, wherein the needle further includes a slider fixedly coupled to the key for sliding the key from the retracted position to the extended position.

23. The system of claim 18, wherein the key includes an expandable volume, wherein the expandable volume extends further in a radially outward direction relative to the needle longitudinal axis in the second position than in the first position.

24. The system of claim 23, wherein the expandable volume includes a bladder in fluid communication with a fluid source,
wherein the expandable volume is movable from the first position to the second position by causing fluid from the fluid source to flow into the bladder,
wherein the needle further includes a slider coupled the fluid source for causing from the fluid source to flow into the bladder.

25. The system of claim 18, wherein the needle includes two or more keys.

26. The system of claim 18, wherein the needle defines one or more radial efflux ports extending from an outer surface of the needle to the lumen.

27. A system for locating an implanted port, the system comprising:
a port comprising:
a reservoir including a membrane and a base coupled to the membrane, the membrane and the base defining a cavity, wherein the reservoir is configured to be implanted under the skin of a person with the membrane facing outwardly from the person; and
a paramagnetic localizing disc disposed in a central portion of the base; and
a port localizer device configured to detect the location of the paramagnetic localizing disc, the port localizer device comprising:
a transmitter/receiver coil defining an opening; and
a detachable handle couplable to and in electrical communication with the transmitter/receiver coil.

28. The system of claim 27, wherein the port further comprises a lock box disposed within the cavity, the lock box defining a lock opening.

29. The system of claim 28, further comprising:
a needle having a needle longitudinal axis and a distal end, the needle defining a lumen extending along the needle longitudinal axis, wherein the needle includes a key disposed within the lumen, the key being movable from a first position to a second position, wherein a portion of the key extends further in a radially outward direction relative to the needle longitudinal axis in the second position than in the first position,
wherein the distal end of the needle is disposable within the lock opening such that the key is couplable to the lock box when in the second position.

30. The system of claim 29, wherein the needle is disposable with the opening of the transmitter/receiver coil.
